(19) United States
(12) Patent Application Publication
(10) Pub. No.: US 2019/0313919 A1
(43) Pub. Date: Oct. 17, 2019

(54) SYSTEM AND METHOD FOR MONITORING ASTHMA SYMPTOMS

(71) Applicant: KONINKLIJKE PHILIPS N.V., ElNDHOVEN (NL)

(72) Inventors: JOHN NIGEL PRITCHARD, LEICESTER (GB); MAREIKE KLEE, STRAELEN (DE); ALBERTUS CORNELIS DEN BRINKER, Eindhoven (NL); NICOLAAS PETRUS WILLARD, VALKENSWAARD (NL); RAINER HILBIG, AACHEN (DE); MARIAN DEKKER, OSS (NL); TAMARA MATHEA ELISABETH NIJSEN, WEERT (NL); HANNEKE HOOGEWERF, GELDERLAND (NL)

(21) Appl. No.: 16/317,195

(22) PCT Filed: Jul. 10, 2017
(86) PCT No.: PCT/EP2017/067204

§ 371 Date: Jan. 11, 2019

(30) Foreign Application Priority Data
Jul. 14, 2016 (EP) 16179396.3

(51) Int. Cl. A61B 5/0205; A61B 5/11; A61B 5/00; G16H 10/20; G16H 50/30

(52) U.S. Cl. CPC A61B 5/0205; A61B 5/1118; A61B 5/4809; A61B 5/0077; A61B 5/024; G16H 10/20; G16H 50/30; A61B 2560/0223; A61B 5/486

(57) ABSTRACT

The present invention relates to a system for monitoring asthma symptoms of a subject (12), comprising a monitoring unit (14) configured to monitor at least one objective asthma symptom of the subject, an analysis unit (16) configured to determine an asthma status of the subject based on the at least one objective asthma symptom monitored by the monitoring unit, a test unit (18) configured to provide a test for collecting subjective asthma symptoms from the subject, if the at least one objective asthma symptom monitored by the monitoring unit (14) has changed in a manner that the change reveals a deterioration of the asthma status of the subject. Further the system comprises a calibration unit (24) configured to calibrate the at least one objective asthma symptom gathered from the subject with respect to a calibration test, wherein the calibration unit (24) is arranged to perform calibration at least once during an initial phase of use of the system (10).

### FIELD OF THE INVENTION

The present invention relates to systems for monitoring asthma symptoms of a subject. The present invention further relates to methods of monitoring asthma symptoms of a subject.

### BACKGROUND OF THE INVENTION

Asthma is an episodic chronic disease that involves disruption of normal respiratory function. Although asthma affects people of all ages, asthma is the most common chronic disorder in childhood. Asthma therapy is required to prevent episodes of extreme worsening of respiratory function that can lead to hospitalization and even death. Asthma therapy requires monitoring of the asthma symptoms of the subject suffering from asthma.

In case of asthmatic children, parents today have typically only subjective information, such as via the opinion of the child and via questionnaires, on the status of their asthmatic child. Observing the child carefully with respect to asthmatic medication usage as well as asthma symptoms, like coughing, wheezing, breathlessness, lower level of physical activity, sleeping problems, and tiredness, is the only possibility for parents of asthmatic children to follow the health status of their child.

Although parents very often recognize in an early phase changes of the health status of the child, e.g. changes in the face, eyes, etc., these changes may be difficult to describe to the doctor without having to show any objective data/insights.

The first issue indicated by parents of asthmatic children is that they cannot always be close to their children for a variety of reasons. Due to obligations, e.g. work, parents may not always be in the same physical location as the child. Also, children growing up are usually less close to their parents, e.g. going to school, visiting friends, going on holidays. As a result, parent cannot follow the children's symptoms close enough to early intervene during health status changes and/or deterioration.

A second issue is that parent often can accurately recall the symptoms for one or two days, but not of the period before that. For example, parents may simply forget or mix up the severity and presence of symptoms. When the health status of the child has changed dramatically, parents organize a visit to the medical doctor.

To judge the health status of an asthmatic subject, clinicians often use an asthma control questionnaire or asthma control test (ACT) to assess the health status of an asthmatic patient, e.g. asthmatic child. This requires the patient or the parent of the child to accurately recall the symptoms of the asthmatic subject at day and night. This is often difficult, especially since these questions often relate to an extended period of several weeks. Parents could use documentations of the symptoms, but this requires additional discipline and is often difficult to fill out, especially in view of the earlier mentioned issues, namely when there is not a too close daily presence of the child to the parent.

This situation is in general not very favorable for parents with an asthmatic child, who want to prevent that the child is coming in a critical asthma attack. Parents want to stay in control of the child's health status and they want to be prepared to have all medication available, which is needed. Besides, the parents of the asthmatic child would like to give correct and objective information to the clinician about the symptoms of the child, which is difficult in the above-described way.

Also for asthmatic adults it is sometimes difficult to follow the symptoms accurately and interact early to prevent any deterioration.

U.S. Pat. No. 8,491,493 B2 discloses a method and system for assessing the health status of a subject, such as the asthma status of a subject, by providing an asthma status score and a confidence rating indicating of the score's reliability using continuous real-time data. The assessment comprises a multi-dimensional analysis in which asthma status scores and respective confidence ratings are generated for multiple individual asthma health dimensions as well as a summary asthma health dimension indicative of overall asthma health.

U.S. Pat. No. 8,758,262 B2 discloses an automated system for monitoring respiratory diseases, such as asthma, which provides non-invasive, multi-model monitoring of respiratory signs and symptoms that can include wheeze and cough. Some embodiments employ a mobile device, such as a cell phone, in which raw data from a microphone and an accelerometer are processed, analyzed, and stored.

US 2015/0242586 A1 discloses a system and method for presenting real-time health information which administers virtual questionnaires and automatically determines a user health status, and presents a visualization to a user indicating a likelihood of a symptom occurrence. The system and method presents results of the analysis to a user to predict health risks, analyze user symptoms, connect users with medical professionals, and provide marketing offers.

US 2013/0024212 A1 discloses a computer-implemented method for providing a questionnaire to a patient based on a patient's current health condition. The method includes measuring physical activity of the patient with an activity monitor, measuring respiration rate with a respiration rate sensor, measuring heart rate with a heart rate monitor, measuring cough frequency with a cough frequency monitor, and executing, on the processor of the computer system, one or more computer program modules configured to generate a questionnaire to gather information from the patient. The questionnaire comprises a set of questions that are based on the gathered physical activity data, respiration rate data, heart rate data, cough frequency data, or any combination thereof.

Although monitoring of asthma symptoms may be accomplished by the known systems and methods as such, there is still a need to improve such systems and methods.

### SUMMARY OF THE INVENTION

It is an object of the present invention to provide a system and a method for monitoring asthma symptoms of a subject which enable a more accurate and reliable asthma monitoring.

It is a further object of the present invention to provide a system and a method for monitoring asthma symptoms which can assist the asthmatic subject and/or a caregiver, e.g. a parent of an asthmatic child, in identifying particular triggers for the asthma and/or to provide feedback to them of the degree to which the subject's asthma is under control. The invention is defined by the claims.

According to an aspect of the invention, a system for monitoring asthma symptoms of a subject is provided, comprising:

a monitoring unit configured to monitor at least one objective asthma symptom of the subject,

an analysis unit configured to determine an asthma status of the subject based on the at least one objective asthma symptom monitored by the monitoring unit,

a test unit configured to provide a test for collecting subjective asthma symptoms from the subject, if the at least one objective asthma symptom monitored by the monitoring unit has changed in a manner that the change reveals a deterioration of the asthma status of the subject,

a calibration unit configured to calibrate the at least one objective asthma symptom gathered from the subject with respect to a calibration test, wherein the calibration unit is arranged to perform calibration at least once during an initial phase of use of the system.

The system according to the invention has a monitoring unit which is configured to monitor at least one objective asthma symptom of the subject. Objective asthma symptoms are those symptoms which can be physically measured, e.g. by a sensor. Objective asthma symptoms may also be such symptoms which can be input by the subject, or in case of a child, by the parent of the child (a “caregiver”) and which have the character of a fact rather than being subject to a subjective judgment by the subject.

In embodiments of the invention, the at least one objective asthma symptom of the subject may be wheezing, physical activity during day and/or night, coughing, waking up from sleep, which are accessible by a physical measurement by means of one or more sensors.

Further, an objective asthma symptom may be in some embodiments medication usage and/or usage of an asthma action plan. These objective asthma symptoms can be input into the system by the subject or a caregiver.

The objective asthma symptom(s) may be monitored over a period of time, for example days, weeks, months or even years. The monitored objective asthma symptoms may be stored by the system.

The analysis unit of the system according to the invention is configured to determine an asthma status of the subject based on the at least one objective asthma symptom monitored by the monitoring unit. Thus, the system according to the invention enables the subject and/or the caregiver to get continuous insight in the symptoms and makes it possible to follow the symptoms accurately and interact early to prevent any deterioration.

Further, the system according to the invention has a test unit configured to provide a test for collecting subjective asthma symptoms from the subject, if the at least one objective asthma symptom monitored by the monitoring unit has changed in a manner that the change reveals a deterioration of the asthma status of the subject. Thus, the system according to the invention also provides a service with an asthma control test based on subjective asthma symptoms. For example, one uses a test for collecting subjective asthma symptoms in form of a so-called asthma control questionnaire or asthma control test (ACT) to collect the subject information from the subject (see for example http://www.asthmacontroltest.com). The ACT test is a well-accepted patient self-administered questionnaire for identifying those with poorly controlled asthma. The asthma scores resulting from the inputs of the subject or caregiver range from 5 (poor control of asthma) to 25 (complete control of asthma), with higher scores reflecting greater asthma control. An ACT score >19 indicates well-controlled asthma. Preferably, the test unit calculates an asthma score from the collected subjective asthma symptoms.

The system further comprises a calibration unit configured to calibrate the at least one objective asthma symptom gathered from the subject against a calibration test, wherein the calibration unit is arranged to perform calibration at least once during an initial phase of use of the system. Thus, when starting use of the system for the first time, the subject or caregiver fills out the test, for example a questionnaire, and the test unit may calculate from this test an asthma score based on the test, wherein this asthma score is used to initially calibrate the objective asthma symptoms, by using the measured objective asthma symptoms of the first time period, for example the first few weeks of use of the system, as objective reflection of the asthma score calculated from the subjective asthma symptoms. The advantage here is that in the following period, the subject or caregiver does not need to fill out a questionnaire for collecting subjective asthma symptoms, since the monitoring of the objective asthma symptoms now can be used to determine the asthma status of the subject. When the monitoring of the objective asthma symptoms reveals a significant change of the asthma status, the system requests the subject or caregiver to fill out the test to measure the subjective symptoms.

A more detailed example might give further insights. If a subject or patient starts using the system for a period of say 4-5 weeks the patient uses an activity monitor to measure the amount of daily steps. Each day or every other day the patient provides to the test unit the answers to a (standard) asthma questionnaire to determine an asthma status. Preferably one uses a test with a 1- or 2-day recall. Provided sufficient data (for example during at least a few weeks) are collected with statistical analysis a baseline of threshold value for the output of the activity monitor can be determined that decides when the asthma status turns from well controlled to poorly controlled. Alternatively one can statistically determine ranges of poorly controlled, moderately controlled and well controlled asthma. Of course this is just an example. Other objective symptoms one can think of are breath rate, amount of coughing, number of wake-ups during night etc. It is important to note that the calibration unit primarily determines a baseline of threshold value for the objective asthma symptoms and not to calibrate the monitoring unit or a sensor as such. In the following period the patient or caregiver does not need to fill out any questionnaire anymore, while the monitored objective symptoms are used to monitor the status of the patient. When objective symptoms change significantly (in a negative sense) with relation to the baseline, and thus it is expected that the ACT score decreases strongly, a message is sent to the patient or caregiver to fill out the asthma questionnaire again to measure the subjective symptoms, as compared to the objectively measured symptoms.

The system according to the invention thus advantageously uses both, objective and subjective asthma symptoms which provides better assistance to the subject and/or caregiver to get an insight into the asthma symptoms and development thereof in the specific subject, thereby improving asthma control. The system advantageously uses the calibrated objective asthma symptoms of the subject so that collecting subjective asthma symptoms is only done when really needed. Collecting the subjective asthma symptoms in most situations is more time consuming and troublesome for a patient compared to monitoring objective asthma symptoms. The system according to the invention has the advantage that it collects and stores regularly monitored objective and subjective asthma symptoms of the subject, which can be viewed and used by the subject or caregiver and also by a clinician to investigate the health status and scores of the disease and symptoms over weeks/months and even years. The system can also be used for patient documentation/reporting by the clinician.

In a further embodiment, the analysis unit may be configured to determine the asthma status by determining an asthma score based on the at least one objective asthma symptom and/or by comparing a value based on the at least one objective asthma symptom to a threshold value. For example, such a value can be the number of coughs detected during night, the duration of wheezing, the number of wake-ups during night, etc.

In a further embodiment, the test unit may be configured to provide the test as a questionnaire to be filled out by the subject or a caregiver.

In a further embodiment, the test for collecting subjective asthma symptoms can be provided in form of an asthma control questionnaire or asthma control test (ACT) to collect the subject information from the subject (see for example http://www.asthmacontroltest.com).

Differently from the situation prior to the invention, the difficulty with the ACT was that the subject had to remember symptoms over a long time period, such as over 4 weeks. In the present invention, the combination of monitoring at least one objective asthma symptom and providing a test for collecting subjective asthma symptoms has the advantage that asthma control can be based on objective asthma symptoms as well as subjective asthma symptoms. Principally, during long time use of the system, asthma control can be mainly based on monitoring objective asthma symptoms, while collecting subjective asthma symptoms can be done in longer time intervals when the monitoring of an objective asthma symptom reveals a significant change (in the negative sense) related to a baseline or threshold, and thus it is expected that the ACT score decreases strongly. Then, the system sends a message to the subject or caregiver to fill out the asthma control test to measure the subjective symptoms, as compared to the objective symptoms as monitored by the monitoring unit. If the ACT score is, for example, lower than 19, the subject or caregiver is recommended to organize a doctor visit for check-up. If the score of the ACT is not below 19, the subject and/or caregiver are asked to watch careful the objective symptoms and repeat the ACT in, for example, 3 days. If the objective asthma symptoms as monitored by the monitoring unit stay below the baseline or threshold, or the filled out ACT is below 19 after 3 days, the subject or caregiver is recommended to organize a doctor visit for a check-up.

A further advantage of the system according to the invention is that the monitored objective asthma symptoms can be calibrated against the subjective asthma symptoms collected by the test, for example the ACT.

The additional tests for collecting subjective asthma symptoms can be used to further calibrate the scale of the objective asthma symptoms, and after sufficient coverage of all parts of the subjective scales, the objective asthma symptoms which are monitored can be fully calibrated.

Furthermore, with the objective measures and calibration with the subjective input and the ACT score based on the subjective input an objective ACT score may be created.

According to a further embodiment, the calibration test is based on subjective asthma symptoms collected from the same subject whose objective asthma symptoms are monitored.

However, it is also conceivable as provided in a further embodiment, that the calibration test is based on asthma symptom data from other subjects and/or from known relationships between the an objective asthma symptom and subjective asthma symptoms.

In the absence of a full calibration for the particular subject the asthma status of which is monitored, data from comparable patients, and/or general insights or assumptions may be used to create an approximation of the map between objective and subjective asthma symptoms. As an example of the use of general insights, it is known for chronic coughing that the transformation from the objective asthma symptom to the subjective scale roughly follows a logarithmic behaviour with respect to the amount of coughing. Therefore, for not too low amounts of coughing, scale calibration can be obtained from a single point calibration augmented with such knowledge.

The system may further comprise a service for the caregiver, where the usage of the medication is monitored. For example, after 70% of the content of the inhaler seems to be used, the caregiver gets information to take care for new medication.

In further embodiments, the system comprises at least one sensor to measure a quantity indicative of the at least one objective asthma symptom.

The at least one sensor may be chosen from the group comprising a microphone, a movement detector, an activity monitor, an accelerometer, heart rate monitor, breath rate monitor, polymer film sensor, piezoelectric sensor, camera or a medication adherence test-unit. With a microphone, objective asthma symptoms like wheezing, coughing may be monitored, e.g. by algorithms configured to detect wheeze and/or cough in the noise. With an accelerometer or movement detector, objective asthma symptoms like physical activity during day and/or night, waking up from sleep, etc. may be monitored.

In a further embodiment, the system further comprises at least one sensor for sensing environmental air quality. In particular, the sensor may be configured to sense the CO₂ level, the temperature and/or the humidity, and/or particles, and/or volatile organic compounds of the indoor air in the environment of the subject. Based on the air quality, recommendations for actions can be given by the system.

The system preferably further comprises an output unit configured to output, based on the determined asthma status, an advice to take action for improving the asthma status. Preferably the output unit works wirelessly and unobtrusively, for example by using a smart phone (app).

The system may further comprise a monitoring unit, which monitors air quality on the go where the patient can decide to wear a particle/air quality sensor. The unit signals to the subject in case the particle size in the air exceeds a threshold.

The system may further comprise a unit, where local weather/pollen information and/or air pollution information for the subject and the caregiver are made available from open services.

In further embodiments, at least the analysis unit is comprised in a mobile computing device, like a mobile phone or tablet.

The system according to the invention may also comprise a telemonitoring system, where the asthma information measured by the sensors objectively as well as the results from asthma control test are displayed to a caregiver, e.g. on a tablet or screen, so that the caregiver can take action and intervene if necessary.

The telemonitoring system may also comprise a dashboard having a screen on which changes of the objective measures in a negative way are indicated to the clinician by one or more indicators, for example red flags. Also an indicator, e.g. a red flag, may be indicated on the screen, when the ACT score derived from the subjective input from the asthmatic subject is below 19, so that the clinician can intervene and e.g. change medication. Also an indicator, e.g. a red flag, may be given on the screen when the rescue medication usage increases, indicating a deterioration of the asthma status of the subject. Furthermore, the telemonitoring system can be configured to show over a long period no use of maintenance medication so that the clinician or nurse can call and intervene.

Further, the service provided by the system according to the invention may be combined with a call center that follows the data of the subject and contacts the subject or caregiver in case of an indication of deterioration of the subject's asthma status. Furthermore, as mentioned above, with the objective measures and calibration with the subjective input and the ACT score based on the subjective input an objective ACT score may be created. If there is a strong change of this objective ACT score, an indicator, e.g. a red flag may then be given by the system to a caregiver or clinician to check the patient's health status.

The system may further comprise a coaching service that reminds the asthmatic subject or caregiver to take medication, if the medication tracker is indicating that no medication is taken. The coaching may also comprise recommendations to do activities indoor, if there is e.g. pollen alarm.

The service provided by the system may further remind the subject to follow the asthma action plan, and/or give information/educational material on asthma.

In a second aspect of the invention, a method of monitoring asthma symptoms of a subject is provided, comprising:

monitoring at least one objective asthma symptom of the subject,

determining an asthma status of the subject based on the monitoring of the at least one objective asthma symptom,

providing a test for collecting subjective asthma symptoms from the subject, if the at least one objective asthma symptom monitored has changed in a manner that the change reveals a deterioration of the asthma status,

calibrating the at least one objective asthma symptom gathered from the subject against a calibration test, wherein calibrating is performed at least once during an initial phase of use of the system.

It shall be understood that the claimed method has similar and/or identical preferred embodiments as the claimed system and as defined in the dependent claims.

Further preferably, providing the test comprises providing an asthma control questionnaire to be filled out to collect subjective asthma symptoms.

In a third aspect of the present invention, a computer program comprising program code means for causing a computer to carry out the steps of the method according to the second aspect, when said program code means is executed on a computer.

Preferred embodiments of the invention are defined in the dependent claims.

### BRIEF DESCRIPTION OF THE DRAWINGS

These and other aspects of the invention will be apparent from and elucidated with reference to the embodiments described hereinafter. In the following drawings:

FIG. 1 shows a block diagram of units of a system for monitoring asthma symptoms of a subject;

FIG. 2A shows an embodiment of a configuration of at least a part of the system integrated into a mobile computing device in an operation mode of the system;

FIG. 2B shows a further embodiment of the system in a further operation mode;

FIG. 2C shows a further embodiment of the system in a further operation mode;

FIG. 3 shows a further embodiment of the system in a further operation mode;

FIG. 4 shows a further embodiment of the system in a further operation mode; and

FIG. 5 shows a further embodiment of the system in a further operation mode.

### DETAILED DESCRIPTION OF THE INVENTION

In the following, several embodiments of a system for and method of monitoring asthma symptoms of a subject will be described. It is to be understood, that the features of the embodiments which will be described hereinafter can be combined among the embodiments and realized in one and the same system.

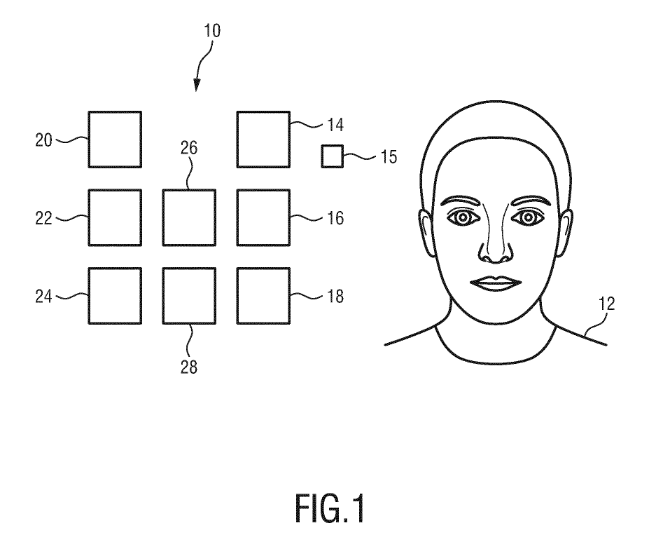

FIG. 1 schematically shows a block diagram of a system 10 for monitoring asthma symptoms of a subject 12. The system 10 comprises several units which will be described hereinafter. Although the units are shown as separate units, it is to be understood that some or all of these units may be integrated into one another, which means that one or more of the units can perform the functions of other units. The units may be configured as hardware, software and/or firmware.

The system 10 comprises a monitoring unit 14. The monitoring unit 14 is configured to monitor at least one objective asthma symptom of the subject 12. An objective asthma symptom is an asthma symptom which can be objectively acquired and is not based on a subjective judgment of a human. In particular, objective asthma symptoms are symptoms which can be measured by a physical measurement, or which can be input as a fact which is not influenced by a subjective judgment of the subject 12 or a caregiver. To this end, the system 10 may comprise one or more sensors 15.

Examples for objective asthma symptoms which can be measured are wheezing, physical activity during day and/or night, coughing, waking up from sleep, heart rate, respiration rate. For example, coughing and wheezing can be measured as sound by a sensor, for example a microphone, wherein coughing and/or wheezing can be detected from the sound by an appropriate algorithm. Further, it is also possible to detect coughing by other means, e.g. a movement sensor in the bed or a wearable respiration monitor. Physical activity can be measured by a sensor, for example a movement detector, in particular by an accelerometer. Thus, the monitoring unit 14 may comprise one or more sensors for measuring the objective asthma symptoms.

Accordingly, the system 10 may comprise sensors, e.g. a microphone by which a wheezing or coughing of the subject 12 can be detected. Further, the system 10 may comprise other or further sensors, e.g. a movement detector, for example based on a polymer film that responds to mechanical stress, which can be arranged under the mattress on which the subject 12 lies during night. Such a movement detector can be used to monitor the physical activity during night and/or waking up of the subject 12 from sleep as well as the heart rate and breathing rate of the subject. A further example for such a sensor is an accelerometer worn by the subject, for example on the hand wrist. Such a sensor is useful for monitoring physical activity of the subject 12 during day. It can also be used to monitor awakening in the night. For awakening in the night, also any other sensor might be used, for example a camera or any other system.

Other objective asthma measures which can be input to the system 10, are medication usage. Furthermore, the system also comprises an asthma action plan.

The system 10 further comprises an analysis unit 16. The analysis unit 16 is configured to determine an asthma status of the subject based on one or more objective asthma symptoms monitored by the monitoring unit 14. The analysis unit 16 can be configured to determine an asthma score representative of the asthma status of the subject 12, based on the monitoring of the one or more objective asthma symptoms by the monitoring unit 14.

Further, the analysis unit 16 may be configured to compare a value based on one or more of the monitored objective asthma symptoms to a threshold value or a baseline. Such a value may be the amplitude of sound of coughing of the subject, the number of coughs, the duration of wheezing in minutes, etc. The analysis unit 16 is configured to detect changes of the monitored objective asthma symptoms over time, and can further analyze whether a change of an objective asthma symptom reveals a deterioration of the asthma status of the subject 12.

Further, the analysis unit 16 can be configured to analyze whether the change of a monitored objective asthma symptom is such significant that the subject 12 or a caregiver should be given an alert indicative of the necessity to take action, for example to visit a doctor.

The system 10 further comprises a test unit 18. The test unit 18 is configured to provide a test for collecting subjective asthma symptoms from the subject. Such a test may be in form of a questionnaire, e.g. an asthma control test (ACT) questionnaire, as will be described later. Subjective asthma symptoms are asthma symptoms which are based on the subjective judgment of the subject 12 or caregiver, and/or which have to be recalled such persons. Further, the test unit 18 is configured to provide the test for collecting subjective asthma systems from the subject, if the one or more objective asthma symptoms monitored by the monitoring unit 14 and analyzed by the analysis unit 16 has changed such, e.g. with respect to a baseline, that the change reveals a deterioration of the asthma status of the subject.

The system 10 further comprises an input unit 20. The input unit 20 can be used by the subject 12 or a caregiver or any other person to input, for example, objective asthma symptoms like medication usage, use of an asthma action plan, or to fill out an asthma test questionnaire.

The system 10 further comprises an output unit 22. The output unit 22 may be configured to output information by sound or graphics. The output unit may be configured as a display, in particular a touch screen. The output unit 22 may be further configured to output, based on the determined asthma condition, an advise to take action by the subject 12 or a caregiver with respect to the asthma condition, for example to visit a doctor or to follow an asthma action plan.

The system 10 may further comprise a calibration unit 24. The calibration unit 24 is configured to calibrate one or more objective asthma symptoms gathered from the subject 12 against a calibration test. Such a calibration test may be based on subjective asthma symptoms collected from the subject 12, for example an asthma control test (ACT) as mentioned above and described later.

The calibration unit 24 is arranged to perform calibration of the one or more objective asthma symptoms at least once during an initial phase of use of the system 10.

The system 10 may further comprise an environmental condition unit 26. The environmental condition unit 26 is configured to provide information for the subject 12 or the caregiver on environmental conditions like air quality (CO₂-level, temperature, humidity, particle concentration, volatile organic compounds), weather information, pollen information, etc.). Such environmental conditions may be measured by the system 10 itself, for example like temperature and humidity, to which end the system may comprise respective sensors, or the information can be provided by external services, in particular internet services (for example weather and/or pollen information, etc.). The environmental condition unit 26 processes this information, and the information may be output by the output unit 22, wherein the system 10 may process the information in order to give an advice to the subject 12 or caregiver regarding asthma control which is output by the output unit 22.

The system 10 may further comprise a storing unit 28 for storing at least one or more objective asthma symptoms gathered from the subject 12. The storing unit 28 may store further information, for example a test for collecting subjective asthma symptoms to be filled out or as filled out by the subject 12 or caregiver, and the like.

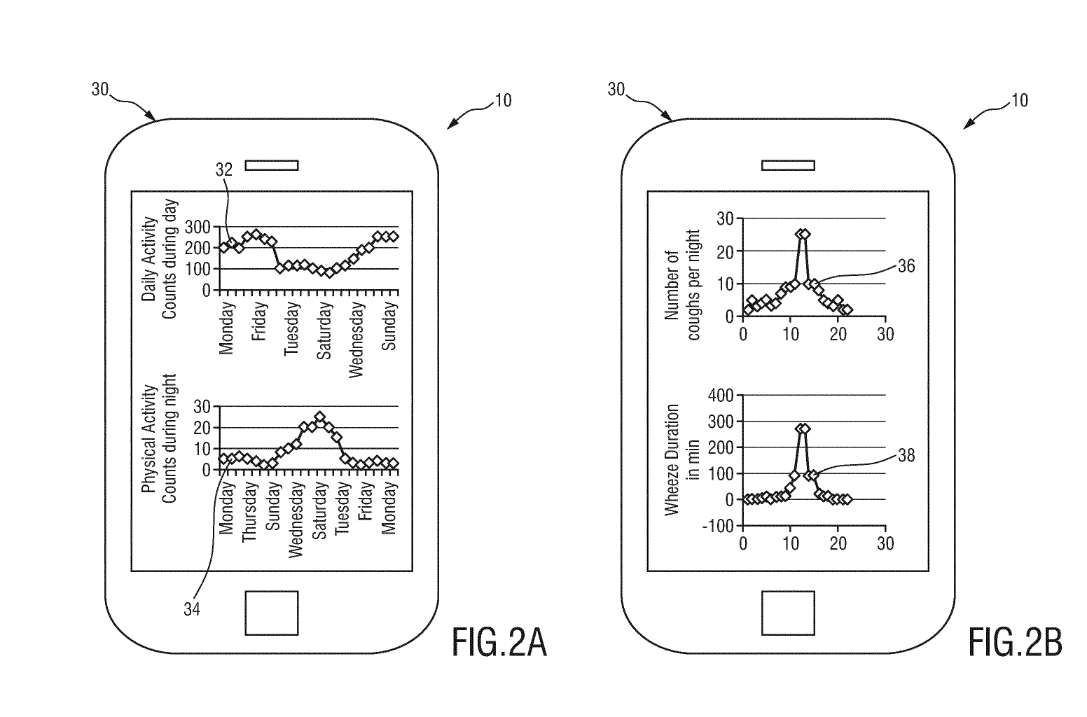

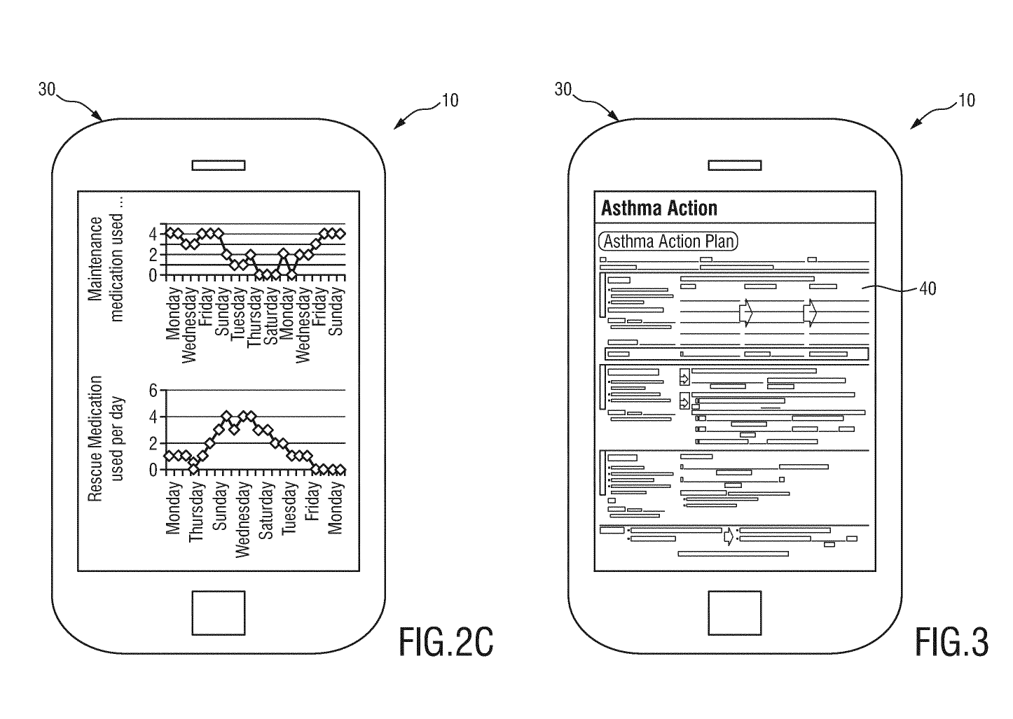

At least some of the units 14-26 of the system may be integrated into a mobile computing device 30, like a mobile phone or tablet, as shown in FIGS. 2A to 2C and FIGS. 3 to 5.

At least the analysis unit 16 may be integrated into a mobile computing device. In addition, the monitoring unit 14, including a microphone and/or a movement detector, the test unit 18, the input unit 20, the output unit 22, the calibration unit 24 and the environmental unit 26 may be integrated into such a mobile computing device.

FIGS. 2A to 2C show an embodiment of the system 10 which at least in part is integrated into a mobile phone 30. In this embodiment of the system 10, the system 10 objectively monitors according to FIG. 2A daily activity during day of the asthmatic subject 12 by activity counts 32. Monitoring the activity counts 32 during day may be accomplished by an accelerometer worn by the subject 12. Further, as shown in FIG. 2A, the system 10 objectively monitors physical activity during night by counts 34 which can be a measure of disturbance of sleep of the subject 12. Activity during night can be measured by an accelerometer worn by the subject 12 or by a movement detector located under the mattress on which the subject 12 lies during night. These sensors can further give information on the number of awakenings in the night as well as other sleep parameters. Sensors under the mattress can also monitor breathing rate and heart rate.

According to FIG. 2B, the system 10 further objectively monitors coughing and wheezing in the night. A curve 36 shows the number of coughs per night as monitored, and a curve 38 shows the duration of wheeze in minutes. Coughing and wheezing can be objectively monitored with a sensor like a microphone. The monitoring unit 14 of the system 10 may be further configured to detect specific coughing, wheezing, or measures the severity of one or both of these phenomena. Further, the monitoring unit 14 of the system 10 can objectively monitor snoring during sleep with a sensor, like a microphone.

Further, the monitoring unit 14 can combine the physical activity during night as monitored and coughing, wheezing and snoring during night is monitored to detect a wakeup of the subject 12 due to asthma or to detect morning asthma symptoms.

According to FIG. 2C, the system 10 further monitors medication usage, for example medication usage on an inhaler with medication tracking. Medication usage is an example of an objective asthma symptom which may be input by the sensor that tracks the usage of maintenance medication and rescue medication, but can also be input by the subject 12 or a caregiver via the input unit 20 into the system 10.

The system 10 according to FIGS. 2A to 2C gives the subject 12 or a caregiver insight into the asthma symptoms. The analysis unit 16 can determine the asthma status of the subject 12 based on the monitoring of one or more of the objective asthma symptoms as shown in FIGS. 2A and 2C.

In a further embodiment, as shown in FIG. 3, the system 10 as shown in FIGS. 2A to 2C, may be further configured to give a feedback to the subject 12 and/or a caregiver based on the asthma status of the subject 12 as determined by the analysis unit 16 based on the monitoring of the objective asthma symptoms by the monitoring unit 14. When the analysis of the monitored objective asthma symptoms as analyzed by the analysis unit 14 reveals that the objective asthma symptoms have changed significantly, for example as exemplary shown in FIGS. 2A to 2C on “Wednesday”, the system 10 recommends the subject 12 or a caregiver to follow an asthma action plan 40 as schematically shown in FIG. 3. The asthma action plan 40 can include information on the type and amount of medicine to be administered to the subject 12 depending on the severity of the asthma status, or, dependent on the severity of the asthma status, the advice to visit a doctor as soon as possible.

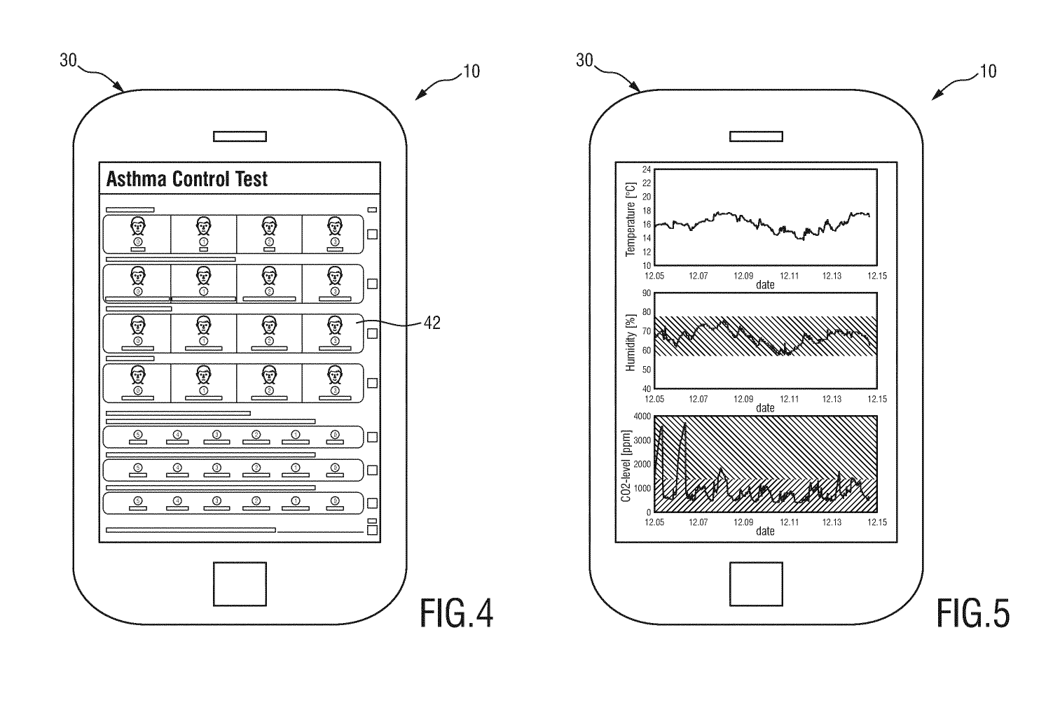

FIG. 4 shows a further embodiment of the system 10 as described so far with respect to FIGS. 2A to 2C and FIG. 3.

As described above, the system 10 objectively monitors objective asthma symptoms of the subject 12 like day activity, night activity/disturbance, night awakenings, coughing/wheezing in the night, and medication usage. As already mentioned above, the test unit 18 is configured to provide a test for collecting subjective asthma symptoms from the subject 12, if the monitored objective asthma systems have changed in a negative sense significantly with respect to a baseline or threshold.

The test provided by the test unit may be configured as an asthma control test (ACT) which is configured as questionnaire 42 as schematically shown in FIG. 4. The asthma control test comprises questions to the subject 12 like “how is your asthma today?”, “how much of a problem is your asthma when you run, exercise or play sports?”, “do you cough because of your asthma?”, and the like, as it is known to a skilled person. The subject 12 or caregiver fills out the questionnaire which is particularly easy if the system 10 is integrated into a mobile phone having a touchscreen.

The test unit 18 calculates from this ACT test an ACT asthma score. Such a test can further be used to calibrate the objective asthma symptoms as monitored by the monitoring unit 14 in an initial phase of use of the system 10. For example, an actual ACT score taken in the first period, for example the first week, of objectively monitoring objective asthma symptoms is used as an objective reflection of the ACT asthma score taken from the ACT test based on subjective asthma symptoms. In the following period, the subject 12 or caregiver does not need to fill out any questionnaire, because monitoring the objective asthma symptoms is sufficient to determine the asthma status of the subject 12. When, however, the objectively monitored objective asthma symptoms, such as medication usage, coughing/wheezing and/or snoring in the night or night disturbance change significantly related to a baseline or threshold, and thus it is expected that the ACT test score decreases strongly, a message is sent to the subject 12 or caregiver by the output unit 22 to fill out the ACT test to measure the subjective symptoms, as compared to the monitored objective symptoms.

If the ACT score calculated from the collected subjective asthma symptoms is lower than nineteen, the subject 12 or caregiver is recommended to organize a doctor visit for check-up.

If the ACT score of the ACT test is not below nineteen, the subject 12 or caregiver is asked to watch carefully the symptoms and repeat the ACT test in, e.g. three days. If the objectively monitored asthma symptoms stay below baseline or the filled out ACT test is below nineteen after three days, the subject 12 or caregiver is recommended to organize a doctor visit for a check-up.

As already mentioned above, in the initial phase of use of the system 10, an ACT test may be performed as a calibration test to calibrate the objectively monitored asthma symptoms. The later additional ACT tests may be used for further calibrating the scale of the objectively monitored asthma symptoms. After sufficient coverage of all parts of the subjective scales, the monitored objective asthma symptoms can be fully calibrated. With the objective measures and calibration with the subjective input and the ACT score based on the subjective input an objective ACT score may be created.

In the absence of a full calibration for the specific subject 12, the calibration test may be based on data from other subjects and/or from relationships between objective asthma symptoms and subjective asthma symptoms. Thus, data from comparable subjects, general insights or assumptions may be used to create an approximation of the map between objective asthma symptoms and subjective asthma symptoms. As an example of the use of general insights: For chronic coughing, it is known that the transformation from the objective asthma symptoms to the subjective scale roughly follows a logarithmic behavior with respect to amount of coughing. Therefore, for not too low amounts of coughing, scale calibration can be obtained from a single point calibration augmented with such knowledge.

In further embodiments of the system 10, the system 10 can track medication usage by the subject 12 through an input of corresponding information by the subject 12 or caregiver. The medication usage can also be tracked by applying a medication tracker on the canister for the inhaled medication. The monitoring unit 14 monitors the medication usage, and, for example, after 70% of the content of the inhaler seems to be used, the subject 12 or caregiver gets information from the output unit 22 to take care for new medication.

In a further embodiment of the system 10, as shown in FIG. 5, the system 10 provides information for the subject 12 or caregiver on environmental conditions by the environmental condition unit 26. As shown in FIG. 5, the system 10 provides information on indoor air quality with respect to temperature, humidity, CO₂-level, particle concentration, volatile organic compounds. Based on the indoor air quality, the system 10 gives recommendations for actions via the output unit 22.

In further embodiments, the system 10 can gather further environmental information, for example from the internet, e.g. local weather information, pollen information, air pollution, and output recommendation to the subject 12 or caregiver, e.g. to do activities only indoor. Further, the system 10 may be configured to enable the subject 12 to decide to wear a particle/air quality sensor, which monitors the air quality on the go. The system 10 give signals to the subject 12 in case the particle concentration in the air exceeds a threshold.

Further, as already described above, the system 10 may be configured to objectively monitor medication usage, activity, night disturbance, night awakening, coughing, wheezing, snoring, heart rate, and/or respiration rate. The monitoring unit 14 may be further configured to detect when maintenance medication is not regularly used. It reminds the subject 12 or caregiver that the subject 12 should use medication.

In the foregoing, also a method of monitoring asthma symptoms of a subject 12 is disclosed, wherein at least one objective asthma symptom of the subject 12 is monitored, an asthma status of the subject 12 based on the monitoring of the at least one objective asthma symptom is determined, and a test for collecting subjective asthma symptoms from the subject is provided, if the monitored at least one objective asthma symptom has changed in a manner that the change reveals a deterioration of the asthma status.

The test provided by the system may be further used as a calibration test for calibrating the objectively measured and monitored asthma symptoms at least once during an initial phase of use of the system 10.

The test may comprise an asthma control questionnaire to be filled out to collect the subjective asthma symptoms.

The system 10 may also comprise a telemonitoring system, where the asthma information measured by the sensors objectively as well as the results from asthma control test are displayed to a caregiver, e.g. on a tablet or screen, so that the caregiver can take action and intervene if necessary. The telemonitoring system may also comprise a dashboard having a screen on which changes of the objective measures in a negative way are indicated to the clinician by one or more indicators, for example red flags. Also an indicator, e.g. a red flag, may be indicated on the screen, when the ACT score derived from the subjective input from the asthmatic subject 12 is below nineteen, so that the clinician can intervene and e.g. change medication. Also an indicator, e.g. a red flag, may be given on the screen when the rescue medication usage increases, indicating a deterioration of the asthma status of the subject. Furthermore, the telemonitoring system can be configured to show over a long period no use of maintenance medication so that the clinician or nurse can call and intervene.

Further, the service provided by the system 10 may be combined with a call center that follows the data of the subject 12 and contacts the subject 12 or a caregiver in case of an indication of deterioration of the subject's asthma status.

Furthermore, as mentioned above, with the objective measures and calibration with the subjective input and the ACT score based on the subjective input an objective ACT score may be created. If there is a strong change of this objective ACT score, an indicator, e.g. a red flag may then be given to a caregiver or clinician to check the patient's health status.

The system 10 may further comprise a coaching service that reminds the asthmatic subject 12 or caregiver to take medication, if the medication tracker is indicating that no medication is taken. The coaching may also comprise recommendations to do activities indoor, if there is e.g. pollen alarm.

The service provided by the system 10 may further remind the subject 12 to follow the asthma action plan, and/or give information/educational material on asthma.

The service provided by the system 10 may further indicate to the subject 12, if the physical activity of the subject 12, measured by the sensor, is exceeding too high values so that there is a risk of an asthma attack due to too high physical activity, to reduce physical activity and slow down.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

A computer program may be stored/distributed on a suitable medium, such as an optical storage medium or a solid-state medium supplied together with or as part of other hardware, but may also be distributed in other forms, such as via the Internet or other wired or wireless telecommunication systems.

Any reference signs in the claims should not be construed as limiting the scope.

1. System for monitoring asthma symptoms of a subject, comprising:
a monitoring unit configured to monitor at least one objective asthma symptom of the subject,
an analysis unit configured to determine an asthma status of the subject based on the at least one objective asthma symptom monitored by the monitoring unit, a test unit configured to provide a test for collecting subjective asthma symptoms from the subject, if the at least one objective asthma symptom monitored by the monitoring unit has changed with relation to a baseline of the at least one objective asthma symptom such that the change reveals a deterioration of the asthma status of the subject,
a calibration unit configured to calibrate the at least one objective asthma symptom gathered from the subject with respect to a calibration test so as to determine the baseline of the at least one objective asthma symptom, wherein the calibration unit is arranged to perform calibration at least once during an initial phase of use of the system.

2. System of claim 1, wherein the analysis unit is configured to determine the asthma status by determining an asthma score based on the at least one objective asthma symptom and/or by comparing a value based on the at least one objective asthma symptom to a threshold value.

3. System of claim 1, wherein the test unit is configured to provide the test as a questionnaire to be filled out by the subject or a caregiver.

4. System of claim 1, wherein the test unit calculates an asthma score from the collected subjective asthma symptoms.

5. System of claim 1, wherein the calibration test is based on subjective asthma symptoms collected from the subject.

6. System of claim 1, wherein the calibration test is based on asthma symptom data from other subjects and/or on known relationships between the at least one objective asthma symptom and subjective asthma symptoms.

7. System of claim 1, wherein the at least one objective asthma symptom is chosen from the group comprising: wheezing, physical activity during day and/or night, coughing, waking up from sleep, medication usage, usage of an asthma action plan, heart rate, respiration rate.

8. System of claim 1, further comprising at least one sensor to measure a quantity indicative of the at least one objective asthma symptom.

9. System of claim 8, wherein the at least one sensor is chosen from the group comprising: microphone, movement detector, accelerometer, polymer film sensor, piezoelectric sensor, camera.

10. System of claim 1, further comprising at least one sensor for sensing environmental air quality.

11. System of claim 1, further comprising an output unit configured to output, based on the determined asthma state, an advice to take action for improving the asthma state.

12. System of claim 1, wherein at least the analysis unit is comprised in a mobile computing device.

13. Method of monitoring asthma symptoms of a subject comprising:
monitoring at least one objective asthma symptom of the subject, determining an asthma status of the subject based on the monitoring of the least one objective asthma symptom,
providing a test for collecting subjective asthma symptoms from the subject, if the at least one objective asthma symptom monitored has changed with relation to a baseline of the at least one objective asthma symptom such that the change reveals a deterioration of the asthma status of the subject,
calibrating the at least one objective asthma symptom gathered from the subject against a calibration test so as to determine the baseline of the at least one objective asthma symptom, wherein calibrating is performed at least once during an initial phase of use of the system.

14. Method of monitoring asthma symptoms of a subject according to claim 13, wherein the calibration is based on subjective asthma symptoms collected from the subject.

15. Computer program comprising program code means for causing a computer to carry out the steps of the method as claimed claim 13, when said program code means is executed on a computer.
